(19) United States
(12) Patent Application Publication
(10) Pub. No.: US 2008/0153122 A1
(43) Pub. Date: Jun. 26, 2008

(54) Method and system for enhancing self-treatment of onychomycosis

(76) Inventors: Susan Beth Cantor, Westfield, NJ (US); Jay Philip Morgan, Collierville, TN (US); R. Stephen Neumann, Basking Ridge, NJ (US); Glenn Goldberg, Nanuet, NY (US); Robert T. Nowak, New Providence, NJ (US); Doreen Frank, Lebanon, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/644,434

(22) Filed: Dec. 21, 2006

(51) Int. Cl. C12Q 1/02; C12M 1/34

(52) U.S. Cl. 435/29; 435/287.1; 514/506; 514/772.3

(57) ABSTRACT

Methods, systems, and devices for medical therapy compliance for the treatment of onychomycosis with a topically applied antifungal agent.

### BACKGROUND

Fungal nail infections (onychomycoses) are caused when a fungus enters the tip or side of the nail and continues to grow under the nail. Fungal nail infections are very common and more often occur in males. Infections can occur due to aggressive nail clipping or nail injury, which provide access for fungus to enter under the nail.

Onychomycoses caused by dermatophytes are the most difficult to treat and contribute greatly to the spread of infectious fungi. All current treatments, whether topical or systemic, require long-term administration of agents, typically 48 weeks or longer of daily administration of topical agents and around 6 months for systemic agents. Failure to diligently follow treatments, for topical agents in particular, can lead to unsuccessful treatment outcome and relapse of the condition. Further, although systemic administration may be preferable to ensure patient compliance, oral administration of fungus-inhibiting agents such as itraconazole and ketoconazole are undesirable due to the potential for side effects of such drugs. In fact, the currently approved orally administered antifungal medications require periodic liver toxicity monitoring.

Topically applied antifungal compositions offer an alternative to orally administered compositions, such as those described in U.S. Pat. No. 4,957,730 (substituted 1-hydroxy-2-pyridone compounds, including ciclopirox); U.S. Pat. No. 6,017,920 (antifungal compositions for external use being retentive in the stratum corneum); U.S. Pat. No. 6,143,794 (antifungal agents having benzylamine moiety applied with excipient to adhere to the surface of a nail so that the antifungal is delivered through the nail plate); U.S. Pat. No. 6,231,875 (acidified nail lacquers containing antifungal); and U.S. Pat. No. 7,094,422 (antifungal agents applied with a nail penetration enhancers). However, the compositions known in the art typically require a prescription and frequent visits to a doctor for nail maintenance to ensure treatment compliance and absence of relapse.

### BRIEF DESCRIPTION OF THE DRAWINGS

FIG. 1 illustrates an example nail growth monitoring device which may be provided as part of an onychomycosis treatment kit, according to an example embodiment of the present invention.

FIG. 1B illustrates another example nail growth monitoring device which may be provided as part of an onychomycosis treatment kit, according to an example embodiment of the present invention.

FIG. 2 illustrates another nail growth monitoring device which may be part of an onychomycosis treatment kit, according to an example embodiment of the present invention.

FIG. 3a illustrates an example group of items that may be included in an onychomycosis treatment kit, according to an example embodiment of the present invention.

FIG. 3b illustrates an example container for an onychomycosis treatment kit, according to an example embodiment of the present invention.

FIG. 4 illustrates an example configuration of a medical therapy compliance management system, according to an example embodiment of the present invention.

FIG. 5 illustrates a flowchart of an example interactive compliance management procedure, according to an example embodiment of the present invention.

### DETAILED DESCRIPTION OF EXAMPLE EMBODIMENTS

It would be useful to have a composition readily available to consumers for use without a prescription for treatment of onychomycosis. To accompany such compositions, it would be useful to provide a method or system to assist in treatment compliance in order to help ensure a successful treatment outcome.

One example embodiment of the present invention provides an article of manufacture which may be used to reduce medication error and enhance therapeutic compliance with use of topical medications for treatment of onychomycosis. The article of manufacture may include (a) a pharmaceutical composition for topical application comprising at least one antifungal agent; (b) indicia for self diagnosis of onychomycosis; (c) nail growth monitoring device; (d) coordinating instructions for application of said antifungal medication(s) in a therapeutic regimen; and (e) a unifying container.

Another example embodiment of the present invention provides a prepackaged unitary dispenser for organizing, storing, instructing, and dispensing topical medicament regimens and enhancing patient compliance. The unitary dispenser may contain (a) at least one topically applied antifungal medication; (b) a device to measure the effectiveness of using the antifungal medication(s); and (c) instructions for coordination of the antifungal medication(s) and device as a treatment regimen.

Another example embodiment of the present invention further provides a nail growth monitoring device.

Another example embodiment of the present invention further provides a medical therapy compliance management system for the treatment of onychomycosis with a topically applied antifungal agent, the system comprising instructions for self-diagnosis of onychomycosis, instructions for proper nail trimming for fungal nails, devices and procedures for tracking growth of healthy nail, and instructions for procedures to prevent relapse.

Another example embodiment of the present invention further provides a medical therapy management system for managing medical therapy to a patient, the system comprising: an interactive system for obtaining data from the patient, wherein the interactive system contains a compliance controller coupled to the interactive system, the compliance controller being configured to receive data from the interactive system, the compliance controller configured to generate notifications and or reports to the patient based upon the data received.

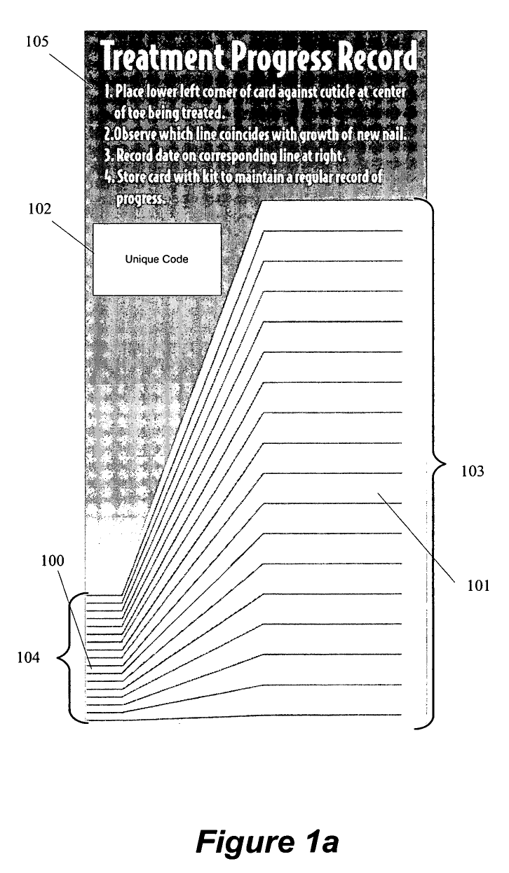

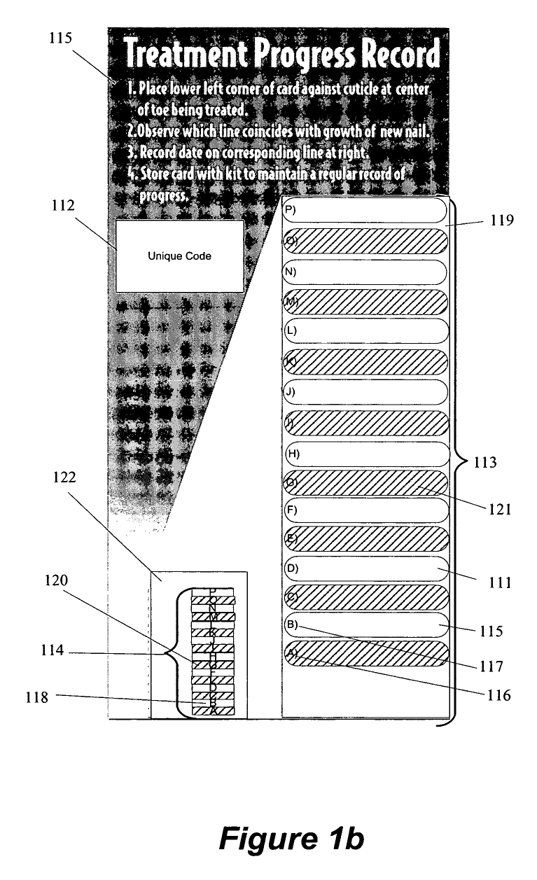

FIG. 1A illustrates an example nail growth monitoring device, according to an example embodiment of the present invention. The example nail growth monitoring device may be provided as part of an onychomycosis treatment kit. In certain embodiments, the monitoring device may be part of a prepackaged kit or dispenser. In one embodiment of the nail growth monitoring device of the invention, the nail growth monitoring device includes a substrate having top, bottom and opposing side edges. Along one side edge a first plurality of horizontal lines 104 may be disposed. The lines 104 may have a first spacing between the lines. A second plurality of horizontal lines 103 may be disposed along the opposite edge. The second plurality of lines may have a second spacing between the lines. The two sets of lines may be correspondingly connected. The first spacing may be narrower than the second spacing. The first set of lines may be disposed so that they correspond to various measurement points. The second set of lines may be more widely spaced, so that they are configured to allow a user to conveniently write information corresponding to the measurement point, e.g., the date when a measured nail has reached the corresponding measurement point.

In certain embodiments, at least a portion of the nail growth monitoring device comprises a material that allows for at least semi-permanent markings to be made. The material that allows for at least semi-permanent markings may be positioned under at least a portion of the second plurality of horizontal lines 103. In additional embodiments, at least a portion of the nail growth monitoring device comprises a transparent or translucent material. In certain embodiments, the transparent or translucent material is positioned under at least a portion of the first plurality of lines 104. The transparent or translucent areas may allow the patient (e.g. the person with the actual nail fungus or a person that is helping to administer or monitor treatment for the person with the nail fungus) to place the nail growth monitoring device over the toenail to aid in tracking treatment progress and healthy nail growth. The treatment tracking area for nail growth may be associated with an area for notation-marking. For example, a single space 100 within the first plurality of horizontal lines 104 may be directly related to a single space 101 within the second plurality of horizontal lines 103. Part of the second plurality of horizontal lines 103 may be made of a material that allows for semi-permanent markings to be made. A patient may then track nail growth and write notes, such as the date, color of the nail, dosage used, topical drug used, etc. Other approaches to distinguishing different associations may be alternately colored spaces, boxed areas, matching numbers or symbols for corresponding measurement points and writing areas, or a combination thereof. Instructions 105 on the use of the nail growth monitoring device may also be displayed on the surface.

A unique code 102 of the patient may also be listed on the nail growth monitoring device. The unique code may be submitted in order to allow the patient to input and track treatment progress and interact with a compliance controller without having to submit contact information. This may be used in circumstances where a user may be too embarrassed to input his or her own contact information. However, in other embodiments, the code may be a blank space to allow a patient to enter a personal contact information which the patient may later use to create a personal treatment page on a website.

The nail growth monitoring device of the invention included as part of the example embodiments is configured to monitor the growth of health nail as a result of treatment. Administration of the antifungal agent according to procedures described in the example embodiments will typically require a period of time, in some instances up to about six months, before the fungus is entirely killed and new, healthy nail can begin to grow. In fungal nail infections, spread of fungus occurs from the tip of the nail towards the cuticle, whereas nail growth occurs in the opposite direction. As the spread of infection is relatively slow (on the order of days and weeks), a subject will be able to determine the extent of fungus growth by a change in color in the nail by a line demarking the boundary between fungal-infected nail and healthy nail. After a period of treatment to kill the fungus, this fungal boundary will cease its progression towards the cuticle and new, healthy nail growth will be seen emanating from cuticle, forcing the fungal boundary towards the nail tip. Typically, the nail growth monitoring device of the included as part of the example embodiments will be used to measure the extent of healthy nail growth, which will increase over time as treatment is continued to completion according to instructions.

FIG. 1B illustrates another alternative example nail growth monitoring device, according to an example embodiment of the present invention. Along one side edge a first set of measurement indicia 114, are provided in the example device as a plurality of horizontal lines. The section of nail growth monitoring device under measurement indicia 114, represented by a rectangular box area 122 may be made of a transparent or translucent material, such as clear plastic, to allow a patient to place the nail directly under the measurement indicia 114. In other example embodiments, particularly, if a transparent material is used, the measurement indicia may be located in different portions of the monitoring device, e.g., at the side rather than the center, or even directly under the writing region, because the transparent monitoring device may be placed directly over the toe rather than adjacent to it when a measurement is taken. In other alternative designs, other sections of the nail growth monitoring device may also be transparent or translucent, or the entire monitoring device may be provided on a single sheet of translucent material such as Lucite, plastic sheeting, or Plexiglas.

The monitoring device may also include a marking area 113 where the user may record the results of measurements taken using the device, e.g., with a marking instrument such as a pen or pencil. The marking area 113 may include a plurality of indicia related to and associated with the measurement indicia 114, which may be disposed in another section of the measurement device, e.g., along the opposite edge from the measurement indicia 114. The marking area 114 may include printing indicating spaces provided for a patient to take notes and monitor treatment progress. e.g., ovals 111 and 115. The individual marking spaces may be distinguished as separate shapes, e.g. ovals, or different colors, or using letters 116 and 1 17 or other symbols. The lines in the transparent measurement section 114 may be correspondingly associated to the plurality of indicia by the use of corresponding letters or symbols, such as 118 associated with 117, or matching colors or shading, such as 120 associated with 121. The letters in the figure are ordered upwards from the bottom of the measurement indicia, to symbolize the direction of the growth to the user; however, other orderings of letters, symbols, or numbers may be used.

The plurality of indicia 113 may also be printed on a detachable sheet 119 that may be connected to the growth monitoring device by an attachment mechanism, such as Velcro, tape, or a removable adhesive layer. This would enable the growth monitoring device to be reusable, or usable over a longer period of time, or used for different toes of the same patient. A unique code 112 of the patient may also be listed on the nail growth monitoring device. Instructions 115 on the use of the nail growth monitoring device may also be displayed on the surface.

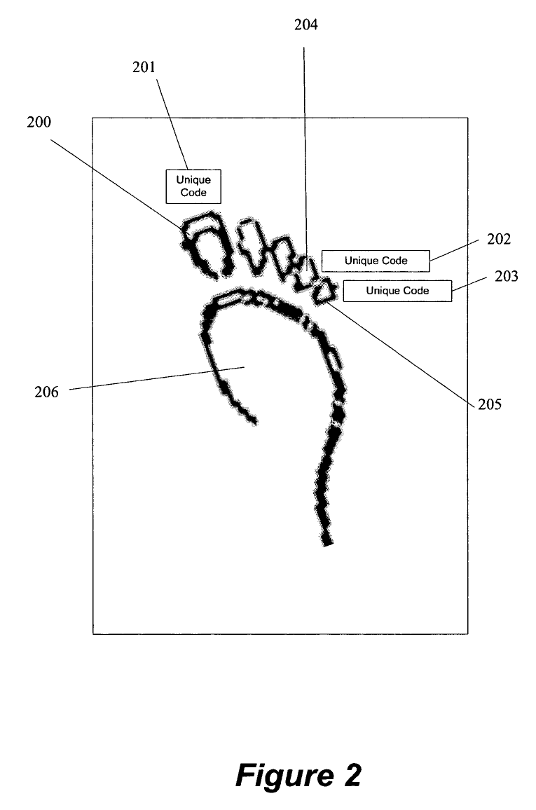

FIG. 2 illustrates a second nail growth monitoring device which is part of an onychomycosis treatment kit, according to an example embodiment of the present invention. A foot 206 may be displayed along with several toes associated with unique code 201, 202, 203 numbers, which are either pre-printed or that can be written in by the patient. The code numbers may correspond to each page of a nail growth monitoring device that contains horizontal lines to measure treatment effects. In the example embodiment, a foot 206 is represented as the right foot; however, a page for the left foot may be a mirror image and may also be monitored in the same way.

A patient may have multiple toe nails that have fungus or onychomycosis. The second nail growth monitoring device may indicate which toes are treated and the nail growth monitoring devices of FIG. 1 may track progress for an individual nail. Multiple copies of the FIG. 1 monitoring device may be provided, one for each treated nail. Alternatively, versions of the FIG. 1 monitoring device can be altered to provide separate tracking areas for multiple toes. Each toe being treated may have a corresponding unique code. For example, the great toe 200 has corresponding unique code 201, the fifth toe 205 may have unique code 203, and the fourth toe 204 may have unique code 202. Each unique code may also be used to monitor treatment on a compliance controller, as will be explained later. In embodiments where a personal contact is provided by the patient, the unique code may be replaced or supplemented with the actual name of the toe, as a default name, or a name input by the patient. This name may then be written in by the patient on the card next to the toe. The unique identifier for each toe in the compliance controller may then be the patient's contact plus the default name or the name provided by the patient.

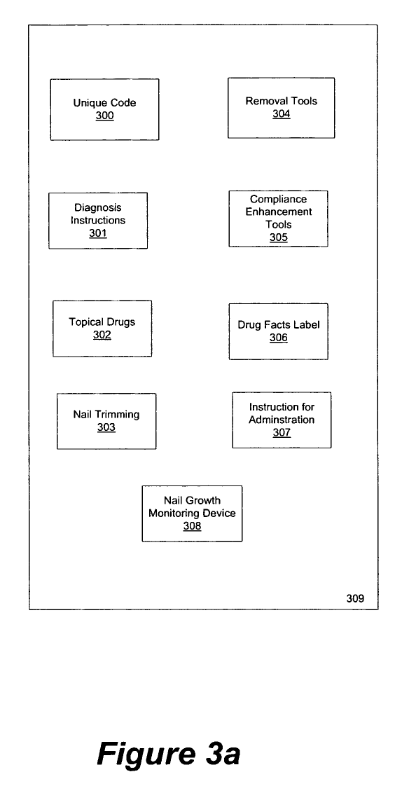

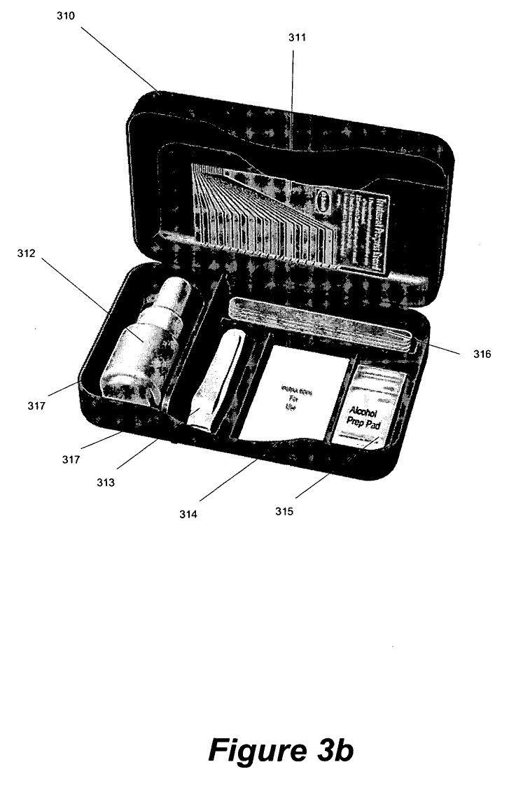

FIG. 3a illustrates an example group of items that may be included in an onychomycosis treatment prepackaged therapeutic device, according to an example embodiment of the present invention. The prepackaged therapeutic device may have a unique code 300 listed on the side of a container 309 or the unique code 300 may be on a paper or label inside the container 309. Use of the unique code will be explained below. In certain embodiments of the prepackaged therapeutic device the indicia for self diagnosis instructions 301 of onychomycosis may be located on a surface of the container 309. This may allow for a consumer to determine whether he or she has onychomycosis and prompting the purchase of the prepackaged therapeutic device. In additional embodiments of the prepackaged therapeutic device, the indicia for self diagnosis of onychomycosis 301 or more detailed explanations may be located inside the container 309. For example, the self-diagnosis instructions may contain a decision tree, a flow chart, interactive table, or may be an aid to be used with a compliance controller as will be explained below.

In certain example embodiments of the prepackaged therapeutic device the coordinating instructions or instructions to administer the drugs 307 are located on a surface of the container. In additional embodiments of the prepackaged therapeutic device, the coordinating instructions 307 are located inside the container 309. In certain example embodiments, the coordinating instructions include instructions on frequency of dosing and number of applications of said topically applied antifungal medication at each dosing. Other instructions may include instructions for proper nail trimming, devices and/or procedures for tracking growth of a nail, instructions for approaches to prevent relapse, and instructions for applying the antifungal agent.

The prepackaged therapeutic device may also contain various kinds of topical drugs 302. Topical drugs may be derived from various types of pharmaceutical formulations, either in individual containers to be applied separately, pre-mixed, or mixed by the patient depending on treatment and dosage. For example, the topical drug may be placed in a six ounce squeeze container for easy application on a nail. The pharmaceutical formulations may include antifungal agents, pharmaceutically-acceptable salts, water-insoluble film-forming agents, physiologically acceptable solvents, or any combination thereof and as explained below.

As used herein, the term “antifungal agent” refers to any compound useful as topical agents to treat fungal infections in animals (including humans) including, but not limited to, fungal infections caused by dermatophytes. Examples of antifungal agents useful in the pharmaceutical formulations of the invention include, but are not limited to, miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, I-ofloxacin, chlortetracycline hydrochloride, oxytetracycline hydrochloride, tetracycline hydrochoride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione and pharmaceutically acceptable salts thereof.

In certain embodiments of the pharmaceutical formulations of the invention, the antifungal agent may be among those described in U.S. Pat. No. 4,957,730, which generally describes 1-hydroxy-2-pyridone antifungals. Examples of certain embodiments of such antifungal compounds include 1-hydroxy-4-methyl-6-n-hexyl, -6-iso-hexyl-, -6-n-heptyl- or -6-iso-heptyl-2-pyridone, 1-hydroxy-4-methyl-6-octyl- or -6-iso-octyl-2-pyridone, in particular as 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-cyclohexylmethyl- or -6-cyclohexyl-ethyl-2-pyridone, the cyclohexyl radical in each case optionally substituted with a methyl radical, 1-hydroxy-4-methyl-6-(2-bicyclo[2,2,1]heptyl)-2-pyridone, 1-hydroxy-3,4-dimethyl-6-benzyl- or -6-dimethylbenzyl-2-pyridone and 1-hydroxy-4-methyl-6-(8-phenyl-ethyl)-2-pyridone.

In certain embodiments the pharmaceutical formulations of this invention include those antifungal agents described in U.S. Pat. No. 6,143,794, which include a benzylamine moiety for example butenafine and related compounds which are also disclosed in U.S. Pat. Nos. 5,021,458 and 5,106,866. Each of the foregoing patents is incorporated by reference. Antifungal compounds of particular interest include, but are not limited to, butenafine and the pharmaceutically-acceptable salts thereof.

It will be appreciated that the amount of pharmaceutically active agent in the topical composition used in the example embodiments will depend on the structure of each active compound, its penetration properties in the nail and its antimicrobial properties.

The pharmaceutically active agent may be present in an amount that is sufficient to produce the desired therapeutic effect when applied according to the recommended regimen. In certain embodiments the pharmaceutically active agent will be present in an amount of 0.01 to 20% by weight, 0.5 to 20%, preferably 2 to 15, percent by weight.

In certain embodiments, the pharmaceutical formulations of the invention may also contain water-insoluble film-forming agent. Examples of suitable film-forming agents include, but are not limited to, substances based on cellulose nitrate or physiologically acceptable polymers, polyvinyl acetate and partially hydrolyzed polyvinyl acetate, copolymers of vinyl acetate such as those containing acrylic acid, crotonic acid, and maleic acid monoalkyl esters, ternary copolymers of vinyl acetate such as those containing crotonic acid and vinyl neodecanoate, or crotonic acid and vinyl propionate, copolymers of methyl vinyl ether and maleic acid monoalkyl esters including maleic acid monobutyl ester, copolymers of fatty acid vinyl esters and acrylic acid or methacrylic acid, copolymers of N-vinyl pyrrolidone, methacrylic acid and methacrylic acid alkyl esters, copolymers of acrylic acid and methacrylic acid or acrylic acid alkyl esters or methacrylic acid alkyl esters, polyvinyl acetals and polyvinyl butyrals, alkyl-substituted poly-N-vinylpyrrolidones, alkyl esters of copolymers of olefins and maleic anhydride and reaction products of colophony with acrylic acid. The alkyl radicals in the esters may be short-chain typically possessing not more than 4 carbon atoms.

Certain embodiments of the pharmaceutical compositions may also include physiologically acceptable solvents, such as hydrocarbons, halogenated hydrocarbons, alcohols, ethers, ketones and esters typically found in topically applied pharmaceutical formulations and cosmetics. Particularly useful solvents include acetic acid esters of monohydric alcohols, such as ethyl and butyl acetate, including those mixed with aromatic hydrocarbons, such as toluene and/or alcohols, such as ethanol or isopropanol. It will be appreciated that the choice of appropriate type and amount of solvent may be based on the desired physical characteristics of the formulation, including drying time, brushability, film thickness and the like.

Certain embodiments of the pharmaceutical formulations may also contain topical formulation additives, meaning additives that are customary in topically applied pharmaceutical formulations and cosmetics, including, but not limited to plasticizers, such as those based on phthalate or camphor, dyestuffs or colored pigments, nacreous agents, sedimentation retarders, sulfonamide resins, silicates, aroma substances, wetting agents, lanolin derivatives, light stabilizers, and substances with a keratolytic and/or keratoplastic action, such as ammonium sulfite, esters and salts of thioglycolic acid, urea, allantoin, enzymes and salicylic acid.

In certain embodiments, the pharmaceutical formulations of the present invention may contain a substance that is retentive in the stratum corneum, examples of which include, but are not limited to, methyl salicylate, glycol salicylate, crotamiton, and peppermint oil or menthol. Such substances may be present in an amount of 0.1 to 10% by weight, preferably 0.5 to 5% by weight, more preferably 1 to 2% by weight, based on the whole weight of the composition.

In certain embodiments, a dermal penetration enhancer may be incorporated into the pharmaceutical formulation to enhance the therapeutic effectiveness of the formulation. Examples of penetration enhancers include, but are not limited to C₈ to C₁₈ alkyl para-aminobenzoate, C₈ to C₁₈ alkyl dimethyl-para-aminobenzoate, C₈ to C₁₈ alkyl cinnamate, C₈ to C₁₈ alkyl methoxycinnamate or C₈ to C₁₈ alkyl salicylate. Specific examples of the dermal penetration enhancer include octyl salicylate, octyl dimethyl para-aminobenzoate or octyl para-methoxycinnamate (Padimate O).

The therapeutic device may also include nail trimming tools 303, such as nail clipper, emery boards, cuticle knives, hoof stick, as well as tools that may help in nail debridement, etc. Tools may also be provided to assist in removal of topical treatment 304, such as alcohol swabs, cotton swabs, etc. Compliance enhancement tools 305 may also be included, such as bathroom mirror stickers, calendars, etc. The nail growth monitoring device 308, such as the examples provided in FIGS. 1 and 2, may also be included. A drug facts label may also be included to explain the ingredients used, in addition to listing the items in the container, and any drug facts that may be required to be listed by law.

FIG. 3b illustrates an example container for an onychomycosis treatment kit, according to an example embodiment of the present invention. A case 310, which may be a hard case as shown but may be in a flexible travel pack, may contain a space for the various accessories of the onychomycosis treatment kit. A nail growth monitoring device 311, such as the example in FIG. 1, may either connected to the case, a removable sheet (as shown), or directly printed onto the case. Various compartments 317 may separate the accessories that may be found in the treatment kit. In the example kit, there may be a bottle containing the particular pharmaceutical composition 312, or a mixture thereof. A nail clipper 313 and nail files 316 may also be in the kit to aid in the grooming of the nail. An alcohol prep pad 315, and other various sterilization devices, may also be found in the kit. Instructions for use 314 may also be provided, and like the nail growth monitoring device, may also be either connected to the case, a removable sheet (as shown), or directly printed onto the case. These accessories and other combinations of accessories may be available in various configurations of a kit depending on the treatment of the nail.

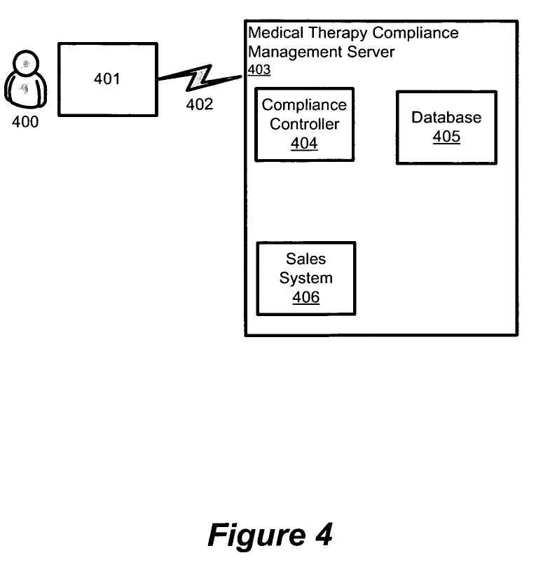

FIG. 4 illustrates an example configuration of a medical therapy compliance management system, according to an example embodiment of the present invention. The patient 400 (e.g. patient, doctor of the patient, patient monitor or patient representative, etc.) may utilize the medical therapy compliance management system on a terminal 100. The system may be either stand-alone, or in additional preferred embodiments, the medical therapy compliance management system may be web-based or accessible over a network, intranet, or internet. In such an instance, the terminal 100 may provide access to the system through a webpage on a computer, a PDA or other handheld device, a wireless cell phone, etc. The term “web-based” is meant to refer to a system that is accessible over internet and/or world wide web portals. The communication medium 402 may be a modem, DSL, cable, Ethernet, wireless, etc.

The medical therapy compliance management system comprises a compliance controller 404 which generates treatment reminders. In certain embodiments the compliance controller 404 may be configured to receive information concerning compliant treatments. In certain preferred embodiments, the compliance controller 404 may be configured to adjust the generation of the reminders based upon compliant treatments recorded. In certain example embodiments, the medical therapy compliance simply sends reminders to ensure that the patient continues the treatment regimen.

In other example embodiments, the medical therapy compliance management system may also have a medical therapy management system, additionally comprising a database 405, wherein the database may store information such as notes made by the patient, treatment results, treatment effects, dosage levels used, pharmaceutical compositions used, patient contact information, and patient physical profile, such as age, race, etc. The database 405 may use this information in order to monitor the patient's progress, interact with the compliance controller 404 to send reminders to the patient, and adjust the treatment plan as needed. The compiled information of all the patients may also be used to improve the dosage and treatment plans and the compliance controller may also utilize the data from the database 405 to generate detailed reports.

In certain embodiments of the medical therapy compliance management system the reminders are sent electronically or through the communication medium 402, for example as electronic voice messages or text messages sent to a phone, blackberry, or other data receiving device. In additional embodiments, the reminders may comprise text messages sent to an e-mail account.

In certain embodiments, the medical therapy compliance management system may also have a sales system 406, which may determine whether a patient may need certain types of pharmaceutical supplements. The patient may place the order from the system, make a recurring order, or the system may automatically send the necessary drugs for treatment to the patient if the patient provided a shipping address.

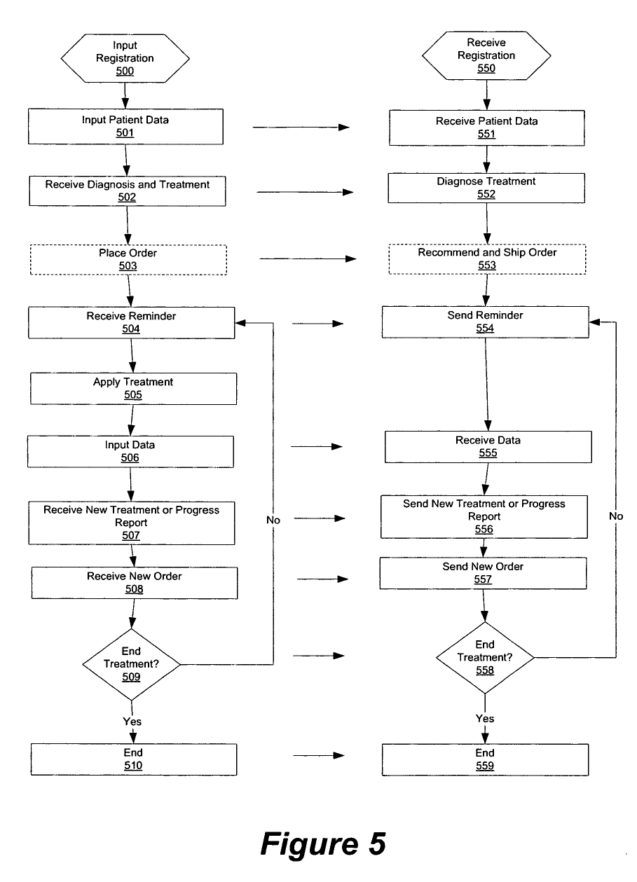

FIG. 5 illustrates a flowchart of an example interactive compliance management procedure, according to an example embodiment of the present invention. The example procedure may include patient registration and tracking. In 500, a patient inputs registration information, either from information from a pre-packaged therapeutic device container, such as the one described in FIG. 3, a nail growth monitoring device, or simply the patient's own contact information, such as a phone number, e-mail, etc. Once the patient has been registered, in 501, the patient may input additional patient data, to create a patient profile. Patient data may include patient age, height, allergies to medication, patient caretaker or patient doctor, etc. If the patient has already previously received a pre-packaged therapeutic device, in 502, the patient may receive a diagnosis and treatment plan from the pre-packaged therapeutic device. The patient may then perform a self-diagnosis and initiate treatment.

If the patient had not purchased a pre-packaged therapeutic device, the patient may access the interactive medical therapy compliance management system, and using the data previously entered by the patient, which may have included the toes that have onychomycosis, the degree of injury to the nails, etc., in 502, the patient may receive a diagnosis and treatment from the system.

In 503, if the patient had not received the pre-packaged therapeutic device, after receiving the diagnosis, the patient may receive a recommendation for a pre-packaged therapeutic device that may be best suited for the patient. For example, if the patient has several toes that have onychomycosis the pre-packaged device may contain more of the topical drug than the patient may receive from a pre-packaged therapeutic device that is purchased in a store. The patient may also choose to modify the pre-packaged therapeutic device. For example, the patient may remove certain tools, such as compliance stickers, or nail trimming tools if the patient wants to reduce price. The patient may already have the tools if the patient had bought previous kits, or if the patient wanted to rely on reminders sent from the system rather than using compliance stickers. The tailored pre-packaged therapeutic device may also provide an option of a variety of different types of topical drugs. For example, if the patient is allergic to certain types of creams, alternatives that are available on the system may be provided. The patient may then place an order to purchase the pre-packaged therapeutic device.

In 504, if the patient had not started treatment, the medical therapy compliance management system may track whether the patient has received the shipment and remind the user to start treatment. The patient may adjust how and when to receive reminders. The patient may receive reminders through a text message, e-mail, or voice mail reminder on a data receiving device (e.g. a phone, blackberry, PDA, laptop, etc.). The patient may also choose to send the reminder once a day or several times a day. For example, the patient may choose to receive a voice mail message in the morning at 7 AM when the patient first wakes up, and an e-mail reminder at 11 PM before the patient is going to bed.

In 505, the patient may follow the instructions provided in the pre-packaged therapeutic device or instructions provided from the medical therapy compliance management system, either from the patient logging into the system to receive instructions or provided along with the reminder message. After the patient has applied the treatment, in 506, the patient may update the patient data online and on the nail growth monitoring device. Using the nail growth monitoring device, the patient may use the transparent portion to make notes or indicate treatment effects online. On the system, the date may automatically be recorded, but on the nail growth monitoring system, the patient may have to write the date and results in.

In 507, after inputting the results, if the treatment is not performing as planned, the system may automatically adjust the treatment plan, such as using a higher dose or more frequent treatment, and this may be recommended to the patient. The treatment may be performing on track, and the patient may receive a progress report comparing the expected progress and the patient's progress. If the patient has multiple toes being treated, treatment for one toe may be progressing better than another. Therefore, when reminders are sent, treatment plans may diverge for the different toes. In 508, if a treatment plan is altered or additional medicine is recommended, the patient may receive a recommended order to receive additional drugs or products which the patient may choose to accept or decline.

In 509, if the treatment plan has not ended, the patient would repeat the process of 504 to 508. Otherwise, if the onychomycosis has been eliminated and the treatment is completed, in 510, the patient may stop using the topical drugs.

In the medical therapy compliance management system, in 550 the system may receive registrations from multiple patients or for the same patient but for multiple toes. If a unique code were being used, each profile may be stored as a different foot for the same patient, but the patient may be able to provide a contact information and the unique code numbers in order to consolidate all the reports and treatment under the same patient. In 551, the system would receive patient profile data as input by the patient, and associate data for each toe under the same patient. In 552, the patient may have already self-diagnosed the condition; however, the system may still provide a diagnosis based on the available data provided by the patient. If the patient has not already received a pre-packaged therapeutic device, the system may optionally, in 503, recommend a tailored therapeutic device. The patient may modify the package and the treatment may then be shipped to the patient.

In 554, a compliance controller may determine that the patient is ready to receive a reminder, which may be based on the patient's preferences. The patient may modify reminders, or have the reminders turned off; however, as a default the patient may receive a reminder at a default contact provided by the patient. In 555, the system may receive any updates on the condition of the various toes that are being treated. This information is recorded and in 556 used to either alter the treatment plan or to provide a progress report for the various toes. If the system recommended a new plan, in 557 an order recommendation may also be provided which may include additional supplies or alternative supplies. The system may then send the order if the patient accepts the new treatment plan. If a new plan is not recommended, the patient may still request additional supplies.

In 558, if the system determines that the treatment is complete, in 559 the system may indicate this to the patient and end the process. Otherwise, 554 to 557 may be repeated until the patient's onychomycosis for all the toes has been treated.

Example embodiments that have been described may be encompassed by an article of manufacture combining at least a pharmaceutical composition for topical application, including an antifungal agent, indicia for self diagnosis of onychomycosis, a nail growth monitoring device, coordinating instructions for application of the pharmaceutical composition in a therapeutic regimen for the treatment of onychomycosis, and a unifying container.

The nail growth monitoring device may be made of or printed on a substrate, e.g. paper, cardboard, plastic, etc., and a nail growth measurement device may be coupled to the substrate, the nail growth measurement device including a plurality of measurement levels. A marking region may be coupled to the substrate, the marking region including a plurality of notation-marking subareas respectively associated with the plurality of measurement levels. The notation-marking subareas may be regions separated by horizontal lines spaced apart at fixed increments, or may be also be ovals, or other shapes, as described above. Adjacent notation-marking subareas may also be distinguished by different colors or shades. Horizontal lines separating the notation marking may respectively be connected to lines indicating measurement levels in the measurement device. Spacing between the lines indicating measurement levels in the measurement device may be narrower than the spacing between the horizontal lines separating the marking subareas.

The nail growth monitoring device may comprise a top, bottom and opposing side edges wherein one side edge comprises a first plurality of horizontal lines having a first spacing between the lines and the opposite edge comprises a second plurality of horizontal lines having a second spacing between the lines, wherein the first spacing is narrower than the second spacing, and lines connecting each of the first plurality of horizontal lines to each of the second plurality of horizontal lines. The plurality of lines may not be located on the edge and may instead be located in other areas of the sides. The nail growth monitoring device may also have indicia associating a plurality of toe nails with a corresponding nail growth measurement device and marking region with notation-marking subareas. A portion of the nail growth monitoring device may be made of a material that allows for at least semi-permanent markings to be made. The material may be positioned under at least a portion of the second plurality of horizontal lines. That portion of the nail growth monitoring device may be made of, in part, a transparent or translucent material. The transparent or translucent material may be positioned under at least a portion of the first plurality of lines.

Indicia for self diagnosis of onychomycosis may be printed on a surface of the container or printed on a surface of the container. Coordinating instructions may be printed on a surface of the container or located inside the container. The pharmaceutically active agent provided may be present in an amount of 0.01 to 20% by weight, 0.5 to 20% by weight, or 2 to 15% by weight. Indicia for self-diagnosis may be a flow-chart and accompanying text. Indicia for self-diagnosis may also be an interactive table with accompanying text.

The antifungal agent may be chosen from the group consisting of miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, I-ofloxacin, chlortetracycline hydrochloride, oxytetracycline hydrochloride, tetracycline hydrochoride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione, mixtures thereof, pharmaceutically acceptable salts thereof and mixtures of pharmaceutically acceptable salts thereof. Each of the agents listed, mixtures thereof, pharmaceutically acceptable salts thereof and mixtures of pharmaceutically acceptable salts thereof are equally preferred specific example embodiments of the invention described herein.

The pharmaceutical composition may further contain a water insoluble film-forming substance, such as from the group consisting of cellulose nitrate, polyvinyl acetate, partially hydrolyzed polyvinyl acetate, copolymers of vinyl acetate containing acrylic acid, crotonic acid, and maleic acid monoalkyl esters, ternary copolymers of vinyl acetate comprising crotonic acid and vinyl neodecanoate, or crotonic acid and vinyl propionate, copolymers of methyl vinyl ether and maleic acid monoalkyl esters, copolymers of fatty acid vinyl esters and acrylic acid or methacrylic acid, copolymers of N-vinyl pyrrolidone, methacrylic acid and methacrylic acid alkyl esters, copolymers of acrylic acid and methacrylic acid or acrylic acid alkyl esters or methacrylic acid alkyl esters, polyvinyl acetals and polyvinyl butyrals, alkyl-substituted poly-N-vinylpyrrolidones, alkyl esters of copolymers of olefins and maleic anhydride and reaction products of colophony with acrylic acid, and mixtures thereof.

The pharmaceutical formulations may further contain one or more topical formulation additives, such as from the group consisting of plasticizers, dyestuffs, colored pigments, nacreous agents, sedimentation retarders, sulfonamide resins, silicates, aroma substances, wetting agents, lanolin derivatives, light stabilizers, and substances having a keratolytic and/or keratoplastic action. Substances with a keratolytic and/or keratoplastic action may be chosen from the group consisting of ammonium sulfite, esters and salts of thioglycolic acid, urea, allantoin, enzymes and salicylic acid.

Pharmaceutical formulations may further contain one or more substances that are retentive in the stratum corneum present in an amount of 0.1 to 10% by weight, based on the whole weight of the composition, 0.5 to 5% by weight, based on the whole weight of the composition, or 1 to 2% by weight, based on the whole weight of the composition. The substances that are retentive in the stratum corneum may be chosen from the group consisting of methyl salicylate, glycol salicylate, crotamiton, peppermint oil and menthol.

Pharmaceutical formulations may also comprise one or more dermal penetration enhancers chosen from the group consisting of C₈ to C₁₈ alkyl para-aminobenzoate, C₈ to C₁₈ alkyl dimethyl-para-aminobenzoate, C₈ to C₁₈ alkyl cinnamate, C₈ to C₁₈ alkyl methoxycinnamate or C₈ to C₁₈ alkyl salicylate and mixtures there of, or octyl salicylate, octyl dimethyl para-aminobenzoate, octyl para-methoxycinnamate, and mixtures thereof.

An example embodiment may also be an article of manufacture, comprising at least one topically applied antifungal medication, a device to measure the effectiveness of using the antifungal medication, and instructions for coordination of the antifungal medication and the device as a treatment regimen. The coordinating instructions including instructions on frequency of dosing and number of applications of said topically applied antifungal medication at each dosing.

An example embodiment may also be a medical therapy compliance management system for the treatment of onychomycosis with a topically applied antifungal agent. The system may be web-based. The system may comprise instructions describing a self-diagnosis procedure for onychomycosis and means for tracking growth of a nail, provided together with the instructions. The instructions may describe proper nail trimming procedure and may be provided together with the instructions describing the self-diagnosis procedure. The instructions may also describe procedures for preventing onychomycosis relapse, provided together with the instructions describing the self-diagnosis procedure. The instructions may also contain instructions for applying the antifungal agent, provided together with the instructions describing the self-diagnosis procedure. The instructions may also describe proper nail trimming procedure, instructions describing procedures for preventing onychomycosis relapse, instructions for applying the antifungal agent, and instructions describing the self-diagnosis procedure in a pre-printed form. The various combinations of instructions may describe proper nail trimming procedure, instructions describing procedures for preventing onychomycosis relapse, instructions for applying the antifungal agent, and instructions describing the self-diagnosis procedure in a pre-printed form. The instructions described above may be singly, or in combination with the other instructions described above, be provided in a pre-printed or electronic form, or any combination of the two forms thereof.

A compliance controller may be configured to generate treatment reminders and send the generated treatment reminders towards a subject, patient, or caretaker of the patient. The reminders may be sent electronically, or as voice messages or text messages to a data receiving device, such as a PDA, cellular phone, laptop, etc. Reminders may also be sent to an e-mail account. The system may further comprise non-electronic means for reminding the subject to comply with treatment, such as magnets or self-sticking visual aids. The compliance controller may be configured to receive information concerning treatment compliance from a subject. The compliance controller may be configured to adjust the generation of the reminders based upon the received information concerning treatment compliance.

An example embodiment may also be a medical therapy management system for managing medical therapy to a patient. The system may comprise an interactive system configured to obtain data from the patient and a compliance controller coupled to the interactive system, the compliance controller configured to receive data from the interactive system and to generate notifications to the patient based upon the data received from the patient.

An example embodiment may be a nail growth monitoring device comprising a surface with associations between nail growth and notation-marking subareas. The surface may have top, bottom and opposing side edges. The one side edge may represent nail growth comprising a first plurality of horizontal lines having a first spacing between the lines and the opposite edge may contain notation-marking subareas associated with the first side edge and comprises a second plurality of horizontal lines having a second spacing between the lines, wherein the first spacing is narrower than the second spacing, and lines connecting each of the first plurality of horizontal lines to each of the second plurality of horizontal lines. A portion of the device may comprise a material that allows for at least semi-permanent markings to be made. The material may be positioned under at least a portion of the second plurality of horizontal lines A portion of the nail growth monitoring device may comprise a transparent or translucent material. The transparent or translucent material may be positioned under at least a portion of the first plurality of lines.

An example embodiment may be a method of administering a compliance program for nail treatment of onychomycosis, comprising providing instructions for the administration of a treatment of a nail growth condition based on a diagnosis of the nail growth condition, providing a nail growth monitoring device, providing a pharmaceutical composition for topical application comprising at least one antifungal agent, registering a patient with a medical therapy compliance management system, and updating patient information on the medical therapy compliance management system.

The diagnosis may be a self-diagnosis, may be determined by a medical therapy compliance management system based on input from a patient or input by a health care professional. The nail growth monitoring device may further comprise a substrate, a nail growth measurement device coupled to the substrate, the nail growth measurement device including a plurality of measurement levels, a marking region coupled to the substrate, the marking region including a plurality of notation-marking subareas respectively associated with the plurality of measurement levels. The notation-marking subareas may be horizontal lines, or other shapes, spaced apart at fixed increments. The notation-marking subareas are differently colored.

The nail growth monitoring device may further comprise indicia associating a plurality of toe nails with a corresponding nail growth measurement device and marking region with notation-marking subareas. The nail growth monitoring device may be provided over a network, within a pre-packaged device, or both.

The antifungal agent may be chosen from the group consisting of miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, I-ofloxacin, chlortetracycline hydrochloride, oxytetracycline hydrochloride, tetracycline hydrochoride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione, mixtures thereof, pharmaceutically acceptable salts thereof and mixtures of pharmaceutically acceptable salts thereof.

The pharmaceutical composition may further comprise a water insoluble film-forming substance, one or more topical formulation additives, one or more substances that are retentive in the stratum corneum, or one or more dermal penetration enhancer. The pharmaceutical composition may be chosen by a doctor or determined by the medical therapy compliance management system based on input from the patient.

The patient may be registered with a code provided on the nail growth monitoring device. The patient may be registered with a personal contact. Reminders may be sent to a patient's personal contact. Instructions may be modified for the administration of the nail growth condition, wherein the modifications may be based on progress input by the patient.

An example embodiment may be a nail growth monitoring device comprising a substrate, a nail growth measurement device coupled to the substrate, the nail growth measurement device including a plurality of measurement levels, a marking region coupled to the substrate, the marking region including a plurality of notation-marking subareas respectively associated with the plurality of measurement levels.

The notation-marking subareas may be regions separated by horizontal lines spaced apart at fixed increments. Adjacent notation-marking subareas may be differently colored. The horizontal lines separating the notation marking are respectively connected to lines indicating measurement levels in the measurement device. Spacing between the lines indicating measurement levels in the measurement device may be narrower than the spacing between the horizontal lines separating the marking subareas.

The nail growth monitoring device may comprise top, bottom and opposing side edges wherein one side edge comprises a first plurality of horizontal lines having a first spacing between the lines and the opposite edge comprises a second plurality of horizontal lines having a second spacing between the lines, wherein the first spacing is narrower than the second spacing, and lines connecting each of the first plurality of horizontal lines to each of the second plurality of horizontal lines. The nail growth monitoring device further comprises indicia associating a plurality of toe nails with a corresponding nail growth measurement device and marking region with notation-marking subareas. A portion of the nail growth monitoring device comprises a material that allows for at least semi-permanent markings to be made. The material may positioned under at least a portion of the second plurality of horizontal lines. A portion of the nail growth monitoring device may comprises a transparent or translucent material.

### Modifications

In the preceding specification, the present invention has been described with reference to specific example embodiments thereof. It will, however, be evident that various modifications and changes may be made thereunto without departing from the broader spirit and scope of the present invention as set forth in the claims that follow. The specification and drawings are accordingly to be regarded in an illustrative rather than restrictive sense.

What is claimed is:

1. An article of manufacture, comprising:
a pharmaceutical composition for topical application comprising at least one antifungal agent;
indicia for self diagnosis of onychomycosis;
a nail growth monitoring device;
coordinating instructions for application of said pharmaceutical composition in a therapeutic regimen for the treatment of onychomycosis; and
a unifying container.

2. The article of manufacture of claim 1, wherein the nail growth monitoring device further comprises:
a substrate;
a nail growth measurement device coupled to the substrate, the nail growth measurement device including a plurality of measurement levels;
a marking region coupled to the substrate, the marking region including a plurality of notation-marking subareas respectively associated with the plurality of measurement levels.

3. The article of manufacture of claim 2, wherein the notation-marking subareas are regions separated by horizontal lines spaced apart at fixed increments.

4. The article of manufacture of 2, wherein adjacent notation-marking subareas are differently colored.

5. The article of manufacture of claim 3, wherein the horizontal lines separating the notation marking are respectively connected to lines indicating measurement levels in the measurement device.

6. The article of manufacture of claim 5, wherein the spacing between the lines indicating measurement levels in the measurement device is narrower than the spacing between the horizontal lines separating the marking subareas.

7. The article of manufacture of claim 1, wherein the nail growth monitoring device comprises top, bottom and opposing side edges wherein one side edge comprises a first plurality of horizontal lines having a first spacing between the lines and the opposite edge comprises a second plurality of horizontal lines having a second spacing between the lines, wherein the first spacing is narrower than the second spacing, and lines connecting each of the first plurality of horizontal lines to each of the second plurality of horizontal lines.

8. The article of manufacture of claim 1, wherein the nail growth monitoring device further comprises indicia associating a plurality of toe nails with a corresponding nail growth measurement device and marking region with notation-marking subareas.

9. The article of manufacture of claim 1, wherein at least a portion of the nail growth monitoring device comprises a material that allows for at least semi-permanent markings to be made.

10. The article of manufacture of claim 9, wherein said material is positioned under at least a portion of the second plurality of horizontal lines.

11. The article of manufacture of claim 2, wherein at least a portion of the nail growth monitoring device comprises a transparent or translucent material.

12. The article of manufacture of claim 11, wherein the transparent or translucent material is positioned under at least a portion of the first plurality of lines.

13. The article of manufacture of claim 1, wherein the indicia for self diagnosis of onychomycosis are printed on a surface of the container.

14. The article of manufacture of claim 1, wherein the coordinating instructions are printed on a surface of the container.

15. The article of manufacture of claim 1, wherein the indicia for self diagnosis of onychomycosis are located inside the container.

16. The article of manufacture of claim 1, wherein the coordinating instructions are located inside the container.

17. The article of manufacture of claim 1, wherein the pharmaceutically active agent is present in an amount of 0.01 to 20% by weight.

18. The article of manufacture of claim 1, wherein the pharmaceutically active agent is present in an amount of 0.5 to 20% by weight.

19. The article of manufacture of claim 1, wherein the pharmaceutically active agent is present in an amount of 2 to 15% by weight.

20. The article of manufacture of claim 1, wherein the antifungal agent is chosen from the group consisting of miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, I-ofloxacin, chlortetracycline hydrochloride, oxytetracycline hydrochloride, tetracycline hydrochoride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione, mixtures thereof, pharmaceutically acceptable salts thereof and mixtures of pharmaceutically acceptable salts thereof.

21. The article of manufacture of claim 1, wherein the pharmaceutical composition further comprises a water insoluble film-forming substance.

22. The article of manufacture of claim 21, wherein the water insoluble film-forming substance comprises a substance chosen from the group consisting of cellulose nitrate, polyvinyl acetate, partially hydrolyzed polyvinyl acetate, copolymers of vinyl acetate containing acrylic acid, crotonic acid, and maleic acid monoalkyl esters, ternary copolymers of vinyl acetate comprising crotonic acid and vinyl neodecanoate, or crotonic acid and vinyl propionate, copolymers of methyl vinyl ether and maleic acid monoalkyl esters, copolymers of fatty acid vinyl esters and acrylic acid or methacrylic acid, copolymers of N-vinyl pyrrolidone, methacrylic acid and methacrylic acid alkyl esters, copolymers of acrylic acid and methacrylic acid or acrylic acid alkyl esters or methacrylic acid alkyl esters, polyvinyl acetals and polyvinyl butyrals, alkyl-substituted poly-N-vinylpyrrolidones, alkyl esters of copolymers of olefins and maleic anhydride and reaction products of colophony with acrylic acid, and mixtures thereof.

23. The article of manufacture of claim 1, wherein the pharmaceutical formulations further contain one or more topical formulation additives.

24. The article of manufacture of claim 23, wherein the one or more topical formulation additives are chosen from the group consisting of plasticizers, dyestuffs, colored pigments, nacreous agents, sedimentation retarders, sulfonamide resins, silicates, aroma substances, wetting agents, lanolin derivatives, light stabilizers, and substances having a keratolytic and/or keratoplastic action.

25. The article of manufacture of claim 24, wherein the substances with a keratolytic and/or keratoplastic action are chosen from the group consisting of ammonium sulfite, esters and salts of thioglycolic acid, urea, allantoin, enzymes and salicylic acid.

26. The article of manufacture of claim 1, wherein the pharmaceutical formulations further comprise one or more substances that are retentive in the stratum corneum.

27. The article of manufacture of claim 26, wherein the one or more substances that are retentive in the stratum corneum are present in an amount of 0.1 to 10% by weight, based on the whole weight of the composition.

28. The article of manufacture of claim 26, wherein the one or more substances that are retentive in the stratum corneum are present in an amount of 0.5 to 5% by weight, based on the whole weight of the composition.

29. The article of manufacture of claim 26, wherein the one or more substances that are retentive in the stratum corneum are present in an amount of 1 to 2% by weight, based on the whole weight of the composition.

30. The article of manufacture of claim 26, wherein the substance(s) that are retentive in the stratum corneum are chosen from the group consisting of methyl salicylate, glycol salicylate, crotamiton, peppermint oil and menthol.

31. The article of manufacture of claim 1, wherein the pharmaceutical formulations further comprise one or more dermal penetration enhancer.

32. The article of manufacture of claim 31, wherein the dermal penetration enhancer is chosen from the group consisting of C₈ to C₁₈ alkyl para-aminobenzoate, C₈ to C₁₈ alkyl dimethyl-para-aminobenzoate, C₈ to C₁₈ alkyl cinnamate, C₈ to C₁₈ alkyl methoxycinnamate or C₈ to C₁₈ alkyl salicylate and mixtures there of.

33. The article of manufacture of claim 32, wherein the dermal penetration enhancer is chosen from the group consisting of octyl salicylate, octyl dimethyl para-aminobenzoate, octyl para-methoxycinnamate, and mixtures thereof.

34. The article of manufacture of claim 1, wherein the indicia for self-diagnosis is a flow-chart and accompanying text.

35. The article of manufacture of claim 1, wherein the indicia for self-diagnosis is an interactive table with accompanying text.

36. An article of manufacture, comprising:
(a) at least one topically applied antifungal medication;
(b) a device to measure the effectiveness of using the antifungal medication(s); and
(c) instructions for coordination of the antifungal medication and the device as a treatment regimen.

37. The article of manufacture of claim 36, wherein said coordinating instructions including instructions on frequency of dosing and number of applications of said topically applied antifungal medication at each dosing.

38. A medical therapy compliance management system for the treatment of onychomycosis with a topically applied antifungal agent, the system comprising:
instructions describing a self-diagnosis procedure for onychomycosis; means for tracking growth of a nail, provided together with said instructions.

39. The system of claim 38, further comprising:
instructions describing proper nail trimming procedure, provided together with said instructions describing the self-diagnosis procedure.

40. The system of claim 38, further comprising instructions describing procedures for preventing onychomycosis relapse, provided together with said instructions describing the self-diagnosis procedure.

41. The system of claim 38, further comprising instructions for applying the antifungal agent, provided together with said instructions describing the self-diagnosis procedure.

42. The system of any of claim 39, 40 and 41, wherein at least one of said instructions describing proper nail trimming procedure, instructions describing procedures for preventing onychomycosis relapse, instructions for applying the antifungal agent, and instructions describing the self-diagnosis procedure are provided in a pre-printed form.

43. The system of any of claim 39, 40 and 41, wherein all of said instructions describing proper nail trimming procedure, instructions describing procedures for preventing onychomycosis relapse, instructions for applying the antifungal agent, and instructions describing the self-diagnosis procedure are provided in a pre-printed form.

44. The system of any of claim 39, 40 and 41, wherein at least one of said instructions describing proper nail trimming procedure, instructions describing procedures for preventing onychomycosis relapse, instructions for applying the antifungal agent, and instructions describing the self-diagnosis procedure are provided electronically.

45. The system of any of claim 39, 40 and 41, wherein all of said instructions describing proper nail trimming procedure, instructions describing procedures for preventing onychomycosis relapse, instructions for applying the antifungal agent, and instructions describing the self-diagnosis procedure are provided electronically.

46. The system of claim 38, further comprising:
a compliance controller configured to generate treatment reminders and send the generated treatment reminders towards a subject.

47. The system of claim 38, further comprising:
a compliance controller configured to receive information concerning treatment compliance from a subject.

48. The system of claim 47 wherein the compliance controller is configured to adjust the generation of the reminders based upon the received information concerning treatment compliance.

49. The system of claim 38 that is web-based.

50. The system of claim 46, wherein the reminders are sent electronically.

51. The system of claim 46, wherein the reminders comprise voice messages sent to a data receiving device.

52. The system of claim 46, wherein the reminders comprise text messages sent to a data receiving device.

53. The system of claim 46, wherein the reminders are sent to an e-mail account.

54. The system of claim 38 further comprising:
non-electronic means for reminding the subject to comply with treatment

55. The system of claim 54, wherein the non-electronic means comprise at least one of magnets or self-sticking visual aids.

56. A medical therapy management system for managing medical therapy to a patient, the system comprising:
an interactive system configured to obtain data from the patient; and
a compliance controller coupled to the interactive system, the compliance controller configured to receive data from the interactive system and to generate notifications to the patient based upon the data received from the patient.

57. A nail growth monitoring device comprising a surface with associations between nail growth and notation-marking subareas.

58. The nail growth monitoring device of claim 57, wherein the surface has top, bottom and opposing side edges.

59. The nail growth monitoring device of claim 58, wherein one side edge represents nail growth comprising a first plurality of horizontal lines having a first spacing between the lines and the opposite edge contains notation-marking subareas associated with the first side edge and comprises a second plurality of horizontal lines having a second spacing between the lines, wherein the first spacing is narrower than the second spacing, and lines connecting each of the first plurality of horizontal lines to each of the second plurality of horizontal lines.

60. The nail growth monitoring device of claim 57, wherein at least a portion of the device comprises a material that allows for at least semi-permanent markings to be made.

61. The nail growth monitoring device of claim 60, wherein said material is positioned under at least a portion of the second plurality of horizontal lines.

62. The nail growth monitoring device of claim 57, wherein at least a portion of the nail growth monitoring device comprises a transparent or translucent material.

63. The nail growth monitoring device of claim 62, wherein the transparent or translucent material is positioned under at least a portion of the first plurality of lines.

64. A method of administering a compliance program for nail treatment of onychomycosis, comprising:
providing instructions for the administration of a treatment of a nail growth condition based on a diagnosis of the nail growth condition;
providing a nail growth monitoring device;
providing a pharmaceutical composition for topical application comprising at least one antifungal agent;
registering a patient with a medical therapy compliance management system; and
updating patient information on the medical therapy compliance management system.

65. The method of claim 64, wherein the diagnosis is a self-diagnosis.

66. The method of claim 64, wherein the diagnosis is determined by a medical therapy compliance management system based on input from a patient.

67. The method of claim 64, wherein the diagnosis is input by a health care professional.

68. The method of claim 64, wherein the nail growth monitoring device further comprises:
a substrate;
a nail growth measurement device coupled to the substrate, the nail growth measurement device including a plurality of measurement levels;
a marking region coupled to the substrate, the marking region including a plurality of notation-marking subareas respectively associated with the plurality of measurement levels.

69. The method of claim 68, wherein the notation-marking subareas are horizontal lines spaced apart at fixed increments.

70. The method of claim 68, wherein the notation-marking subareas are differently colored.

71. The method of claim 68, wherein the nail growth monitoring device further comprises indicia associating a plurality of toe nails with a corresponding nail growth measurement device and marking region with notation-marking subareas.

72. The method of claim 68, wherein the nail growth monitoring device is provided over a network.

73. The method of claim 68, wherein the nail growth monitoring device is provided within a pre-packaged device.

74. The method of claim 64, wherein the antifungal agent is chosen from the group consisting of miconazole, econazole, ketoconazole, itraconazole, fluconazole, bifoconazole, terconazole, butoconazole, tioconazole, oxiconazole, sulconazole, saperconazole, clotrimazole, isoconazole, butoconazole, clioquinol, lanoconazole, neticonazole, ciclopirox, butenafine, undecylenic acid, haloprogin, tolnaftate, nystatin, ciclopirox olamine, terbinafine, amorolfine, naftifine, elubiol, griseofulvin, corticosteroids, amphotericin, calcipotriene, anthraline, minoxidil, minoxidil sulfate, retinoids, cysteine, acetyl cysteine, methionine, glutathione, biotin, finasteride and ethocyn, tea tree oil, mupirocin, neomycin sulfate bacitracin, polymyxin B, I-ofloxacin, chlortetracycline hydrochloride, oxytetracycline hydrochloride, tetracycline hydrochoride, clindamycin phosphate, gentamicin sulfate, benzalkonium chloride, benzethonium chloride, hexylresorcinol, methylbenzethonium chloride, phenol, quaternary ammonium compounds, triclocarbon, triclosan, flucytosine, salicylic acid, fezatione, ticlatone, triacetin, zinc pyrithione and sodium pyrithione, mixtures thereof, pharmaceutically acceptable salts thereof and mixtures of pharmaceutically acceptable salts thereof.

75. The method of claim 64, wherein the pharmaceutical composition further comprises a water insoluble film-forming substance.

76. The method of claim 64, wherein the pharmaceutical formulations further contain one or more topical formulation additives.

77. The method of claim 64, wherein the pharmaceutical formulations further comprise one or more substances that are retentive in the stratum corneum.

78. The method of claim 64, wherein the pharmaceutical formulations further comprise one or more dermal penetration enhancer.

79. The method of claim 64, wherein the pharmaceutical composition is chosen by a doctor.

80. The method of claim 64, wherein the pharmaceutical composition is determined by the medical therapy compliance management system based on input from the patient.

81. The method of claim 64, wherein a patient is registered with a code provided on the nail growth monitoring device.

82. The method of claim 64, wherein a patient is registered with a personal contact.

83. The method of claim 64, further comprising sending reminders to a patient's personal contact.

84. The method of claim 64, further comprising modifying instructions for the administration of the nail growth condition, wherein the modifications are based on progress input by the patient.

85. A nail growth monitoring device, comprising:
a substrate;
a nail growth measurement device coupled to the substrate, the nail growth measurement device including a plurality of measurement levels;
a marking region coupled to the substrate, the marking region including a plurality of notation-marking subareas respectively associated with the plurality of measurement levels.

86. The nail growth monitoring device of claim 85, wherein the notation-marking subareas are regions separated by horizontal lines spaced apart at fixed increments.

87. The nail growth monitoring device of 85, wherein adjacent notation-marking subareas are differently colored.

88. The nail growth monitoring device of claim 87, wherein the horizontal lines separating the notation marking are respectively connected to lines indicating measurement levels in the measurement device.

89. The nail growth monitoring device of claim 88, wherein the spacing between the lines indicating measurement levels in the measurement device is narrower than the spacing between the horizontal lines separating the marking subareas.

90. The nail growth monitoring device of claim 85, wherein the nail growth monitoring device comprises top, bottom and opposing side edges wherein one side edge comprises a first plurality of horizontal lines having a first spacing between the lines and the opposite edge comprises a second plurality of horizontal lines having a second spacing between the lines, wherein the first spacing is narrower than the second spacing, and lines connecting each of the first plurality of horizontal lines to each of the second plurality of horizontal lines.

91. The nail growth monitoring device of claim 85, wherein the nail growth monitoring device further comprises indicia associating a plurality of toe nails with a corresponding nail growth measurement device and marking region with notation-marking subareas.

92. The nail growth monitoring device of claim 85, wherein at least a portion of the nail growth monitoring device comprises a material that allows for at least semi-permanent markings to be made.

93. The nail growth monitoring device of claim 92, wherein said material is positioned under at least a portion of the second plurality of horizontal lines.

94. The nail growth monitoring device of claim 85, wherein at least a portion of the nail growth monitoring device comprises a transparent or translucent material.

95. The article of manufacture of claim 20, wherein the antifungal agent is ciclopirox or a pharmaceutically acceptable salt thereof.

96. The article of manufacture of claim 20, wherein the antifungal agent is butenafine or a pharmaceutically acceptable salt thereof.

97. The article of manufacture of claim 20, wherein the antifungal agent is terbinafine or a pharmaceutically acceptable salt thereof.

98. The article of manufacture of claim 36, wherein the antifungal medication is ciclopirox or a pharmaceutically acceptable salt thereof.

99. The article of manufacture of claim 36, wherein the antifungal medication is butenafine or a pharmaceutically acceptable salt thereof.

100. The article of manufacture of claim 36, wherein the antifungal medication is terbinafine or a pharmaceutically acceptable salt thereof.

101. The system of claim 38, wherein the antifungal agent is ciclopirox or a pharmaceutically acceptable salt thereof.

102. The system of claim 38, wherein the antifungal agent is butenafine or a pharmaceutically acceptable salt thereof.

103. The system of claim 38, wherein the antifungal agent is terbinafine or a pharmaceutically acceptable salt thereof.

104. The method of claim 74, wherein the antifungal agent is ciclopirox or a pharmaceutically acceptable salt thereof.

105. The method of claim 74, wherein the antifungal agent is butenafine or a pharmaceutically acceptable salt thereof.

106. The method of claim 74, wherein the antifungal agent is terbinafine or a pharmaceutically acceptable salt thereof.
